(19) United States
(12) Patent Application Publication
(10) Pub. No.: US 2026/0033841 A1
(43) Pub. Date: Feb. 5, 2026

(54) Tourniquet Belt

(71) Applicant: Greg Thompson, Vass, NC (US)

(72) Inventor: Greg Thompson, Vass, NC (US)

(21) Appl. No.: 19/284,229

(22) Filed: Jul. 29, 2025

Related U.S. Application Data
(60) Provisional application No. 63/677,450, filed on Jul. 31, 2024.

(51) Int. Cl. A61B 17/132

(52) U.S. Cl. CPC A61B 17/1322

(57) ABSTRACT

A tourniquet belt is provided that can be used as a conventional belt and as a tourniquet in an emergency. The tourniquet belt comprises a belt strap having an inner layer and an outer layer. A belt buckle at one end of the belt strap engages with a free end of the belt strap to secure the belt strap around a user's waist for non-emergency use as a conventional belt. A tensioning band is disposed between the inner and outer layers of the belt to facilitate use of the belt as a tourniquet. A fastening mechanism is provided for securing the belt strap in an inverted orientation around an appendage for use as a tourniquet. An opening in the inner layer of the belt strap exposes a portion of the tension band when the belt strap is secured around the appendage. A tensioning rod is configured for insertion beneath the exposed portion of the tensioning band and is operable to tighten the tensioning band when the tensioning rod is rotated by the user. A pocket disposed on the inner layer stores the tensioning rod when the tourniquet belt is not being used as a tourniquet.

### RELATED APPLICATIONS

The present application claims benefit of U.S. Provisional Application No. 63/677,450, filed Jul. 31, 2024, the disclosure of which is incorporated herein by reference in its entirety.

### TECHNICAL FIELD

This invention relates to a trauma torniquet that can be worn and function as a belt.

### BACKGROUND

Torniquets can be used in surgical or clinical settings to selectively, temporarily constrict blood flow to a limb or portion of a limb. Tourniquets are also used in a variety of trauma contexts to stop blood loss in limbs, including in emergency field or combat trauma applications by military, emergency medical services, and private citizens. Whether caused by combat or accident, injuries that cause excessive blood loss can result in death if not stopped very quickly—within a few minutes. Thus, a torniquet can be a life-saving device in an emergency trauma situation.

Various effective emergency use torniquets are commercially available. But to be useful, a trauma torniquet must be available at the scene where it is needed within as little as three minutes of the injury. Sometimes, improvised torniquets can be fashioned from items on hand, but too often they are not sufficiently effective.

Military personnel often carry a trauma torniquet with them in combat and are trained in their use. Emergency response personnel are likely to have a trauma torniquet available, but response time may be more than the few-minute window in which it can effectively be used. Some private citizens carry a trauma torniquet with them in preparedness for such an emergency use. But these can be too bulky to carry at all times, so some trained to use them do not carry one or it may be carried inconsistently and get left behind.

Others have proposed the combination of a garment belt with a torniquet to make it more convenient to have a trauma torniquet available in an emergency situation while being convenient and unobtrusively carried. But room for improvement remains.

### SUMMARY

The present invention provides a tourniquet functional as a typical garment belt quickly deployable as an effective trauma torniquet. The tourniquet belt comprises a belt strap having an inner layer and an outer layer. A belt buckle at one end of the belt strap engages with a free end of the belt strap to secure the belt strap around a user's waist for non-emergency use as a conventional belt. A tensioning band is disposed between the inner and outer layers of the belt to facilitate use of the belt as a tourniquet. A fastening mechanism is provided for securing the belt strap in an inverted orientation around an appendage for use as a tourniquet. An opening in the inner layer of the belt strap exposes a portion of the tension band when the belt strap is secured around the appendage. A tensioning rod is configured for insertion beneath the exposed portion of the tensioning band and is operable to tighten the tensioning band when the tensioning rod is rotated by the user. A pocket disposed on the inner layer stores the tensioning rod when the tourniquet belt is not being used as a tourniquet.

In some embodiments, the tourniquet belt further comprises a lanyard attached at each end to the tensioning rod and passing beneath the exposed portion of the tensioning band to retain the tensioning rod.

In some embodiments, the tourniquet belt further comprises a retention tab attached to the inner layer of the belt strap for engagement with the tightening rod after the tourniquet belt is tightened around the appendage to maintain the tension on the tensioning band

In some embodiments, the tourniquet belt further comprises one or more elastic keepers attached to the inner layer to hold the retention tab flat against the inner layer when the tourniquet belt is used as a belt.

In some embodiments, the t fastening mechanism comprises a hook and loop faster attached to the inner layer of the belt strap.

Other aspects, features, benefits, and advantages of the present invention will become apparent to a person of skill in the art from the detailed description of various embodiments with reference to the accompanying drawing figures, all of which comprise part of the disclosure.

### BRIEF DESCRIPTION OF THE DRAWINGS

Like reference numerals are used to indicate like parts throughout the various drawing figures, wherein:

FIG. 1 is an outside plan view of a torniquet belt according to one embodiment of the invention;

FIG. 2 is an inside plan view thereof;

FIG. 3 is a partial pictorial view showing a tensioning rod engaged with a tension band; and

FIG. 4 is a similar view showing the tensioning rod stored in a pocket.

### DETAILED DESCRIPTION

With reference to the drawing figures, this section describes particular embodiments and their detailed construction and operation. Throughout the specification, reference to “one embodiment,” “an embodiment,” or “some embodiments” means that a particular described feature, structure, or characteristic may be included in at least one embodiment. Thus, appearances of the phrases “in one embodiment,” “in an embodiment,” or “in some embodiments” in various places throughout this specification are not necessarily all referring to the same embodiment. Furthermore, the described features, structures, and characteristics may be combined in any suitable manner in one or more embodiments. In view of the disclosure herein, those skilled in the art will recognize that the various embodiments can be practiced without one or more of the specific details or with other methods, components, materials, or the like. In some instances, well-known or elsewhere-described structures, materials, or operations are not shown or not described in detail to avoid obscuring relevant aspects of the embodiments.

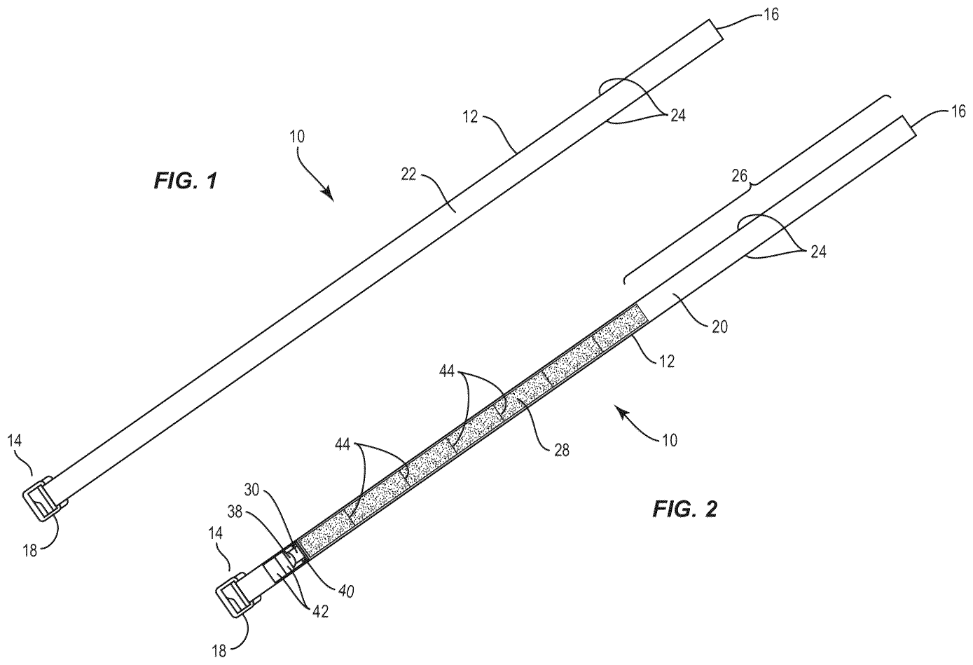

Referring first to FIGS. 1 and 2, therein is shown a torniquet belt device 10 according to one embodiment of the present invention. The device 10 includes an elongated belt body 12 with a fastener end 14 and a free end 16. The fastener end 14 includes a buckle 18 or similar device to adjust the belt body 12 around a user's waist to function as a belt. The belt body 12 preferably is formed of inner and outer elongated layers 20, 22 of material (such as webbing/belting material) that is strong and typically is resistant to stretching. The belt body 12 may typically be about 1.25 to 2 inches in width, with a popular width of about 1.5 inches. The outer and inner layers 20, 22 are secured (such as by sewing, welding, or other means of securely fastening the layers together) along longitudinal edges 24 to form a flat tube with an elongated internal channel or tunnel.

According to one embodiment, the outer layer 22 is the visible component when the tourniquet belt system is worn by a user. The outer layer 22 may have the appearance of an ordinary wearable belt and may include fabric, webbing, leather, synthetic leather, and/or any other suitable component to enable the outer layer to appear similar to an ordinary belt. This enables a user wearing the tourniquet belt system to wear the tourniquet component around the person's waist in a manner similar to that of a person wearing a standard belt without it being noticeable to others or inconvenient to the wearer. The tourniquet parts and features of the device 10 are generally concealed when being worn as a belt. The belt can function as a fashion accessory or as a duty belt. Either can support things typically supported by a belt, such as a holster, equipment, tools, or other accessories.

When a double-ring fastener (buckle 10) is used, substantially all or at least a portion 26 of the inner layer 20 adjacent the free end 16 may be made of the same material as the outer layer 22 so that when fastened there is little or no indication that the device includes a tourniquet feature. This portion 26 would be at least the amount of the belt body 12 that would be expected to show if passed through a ring-type fastener and doubled back to be exposed when worn at the smallest expected size. This aspect may not be important when other types of fastener buckles are used.

The inner side of the body 12 includes features (discussed in more detail below) that may be adjacent the buckle end 14 that allow the device 10 to function as an effective trauma tourniquet. The inner side may include other features that are part of or carried on the inner layer 20. For example, the inner side may include pockets or other features described in more detail below.

The fastener/buckle 18 can be any appropriate type to function as a belt connector, but it can double as a member that allows quick adjustment to significantly smaller circumference when used as a tourniquet. For example, it can be a friction buckle or dual rings (as shown, where the free end is passed first through both rings and then back through only one ring so that when pulled tight around the waist, it remains tight). The length of even a small size adult belt will usually exceed the circumference of an adult thigh.

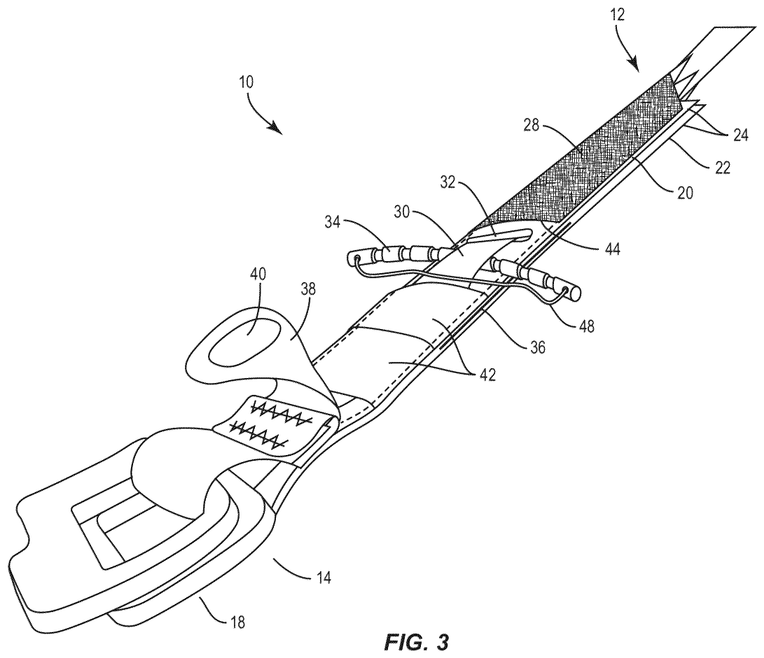

Referring now also to FIG. 3. when used as a trauma tourniquet, the device 10 can be applied to constrict blood flow and stop blood loss from a victim's limb. The belt body is reversed so that the outer layer 22 is situated toward the victim's body. If a double ring fastener (as illustrated) 18 is used, the free end 16 is passed through one or both of the D-shaped rings and can be fastened using hook and loop fastener material to provide preliminary size and tension for tourniquet use. If a different type of belt buckle that does not provide a loop is used, a separate ring (not shown) or similar member can be provided for when the device is used as a trauma tourniquet.

The adjustable attachment can be, for example, by way of standard hook and loop/pile material or a hermaphroditic (dual element) hook and loop fastener material 28 can be used. This dual element material 28 engages with itself without requiring selected placement of either hook or loop material, but does not aggressively attach to other materials, such as clothing, when the device 10 is in use as a belt. Using a hook and loop fastener 28 for torniquet use is preferred over most alternatives because it is easy to use when hands are wet and slippery (such as with the victim's blood, which will be present any time it needs to be used as a trauma torniquet).

For effective constriction, the tourniquet component of the device 10 includes a tourniquet mechanism configured to operate as a tourniquet tensioner to reduce the circumference. According to various embodiments, the tourniquet mechanism includes an elongated fabric strip or band 30, such as a nylon strap or tape. It is noted, however, that any material suitable for use as a tourniquet may be used as the tourniquet layer. The tension band 30 is situated in the channel or tunnel between the inner and outer layers 20, 22 of the belt body 12 and is fixed to the belt body 12 at both ends. It slidably extends through the channel from the buckle end 14 to at least some point along the length of the belt representing at least a significant portion of the required circumference when used as a tourniquet, for example at least 12 inches). The tension band 30 extends through an end or opening 32 in the inner layer 20 so as to have an exposed portion at an area near the buckle end 14. Both ends are fixed in place to the body 12 so that shortening the length of the tension band 30 reduces the circumference of the torniquet loop. One end may extend through or around the buckle 18 with the upper inner and outer layers 20, 22 and be fastened with them (such as by sewing).

A tightening rod 34 is used as a “Spanish windlass” to shorten the tension band 30 by twisting it using the rod 34 as a lever. As used herein, the term “rod” is used nominally and is not meant to define the shape of this member. The rod 34 can be, for example, approximately 4 inches long. It can include a knurled or ribbed surface to enhance grip. Additionally, it can be curved so that when stored in the device 10 it conforms to the curvature of the user's waist when being worn as a belt. A stiffening panel 36 can be positioned under the exposed portion of the tension band 30, such as between the inner and outer belt body layers 20, 22, to maintain shape and prevent injury when the tension band 30 is twisted with the tension rod 34. The panel 36 should be a stiff but bendable material, such as a molded or extruded polymer.

After the rod 34 is used to twist the tension band 30 to shorten the circumference around the limb and stop arterial blood flow/loss, the rod is secured to prevent untwisting. A variety of structures can be used for this purpose, such as the illustrated flexible retention tab 38 secured to the device 10 and having an opening 40 into which an end of the rod 34 can be inserted. The tab 38 can also be a location on which to write the time the tourniquet was applied. The tab 38 can be a strong flexible material that lays flat when not in use and can optionally be secured under one or more elastic keepers 42. The keepers 42 also can allow the tension band 30 to gather when it is twisted with the tensioning rod 34.

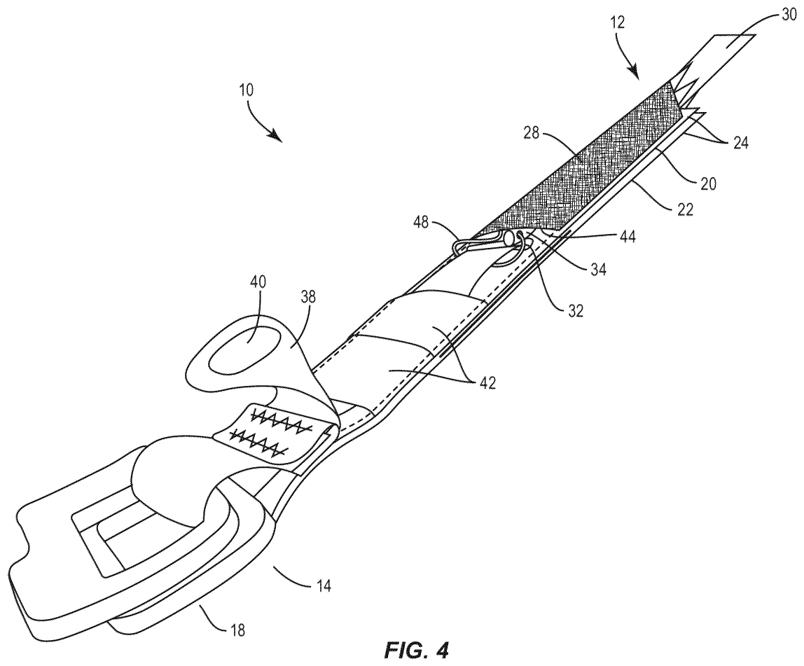

As previously mentioned, the inner side of the belt body 12 may include features such as attachment elements or pockets. Pockets 44 can be, for example, the width of belt by 3-4 inches in length. They can be formed, for example, by segmenting the hook and loop material 28 to form spaced apart opening slots 46. The pockets 44 can be used to store, for example, the tension rod 34 as shown in FIG. 4. Or (not shown) a handcuff key, a writing instrument, a USB drive or other data storage device, medical or other information, and/or an indicator of wearer's blood type can be stored. Longer items, like restraining cuff ties (zip ties) or a saw blade, can be held by multiple pocket segments 44. Such stored items do not interfere with the device's immediate use as a trauma tourniquet.

The tensioning rod 34 can be retained with a lanyard or a loop 48 that extends from one end to the other, as shown in FIG. 3. This prevents the rod 34 form becoming separated from the device 10 and lost, especially when in the presence of blood. The lanyard or loop 48 can be longer than the rod 34 to allow the rod 34 to be tucked into an adjacent pocket 44 for storage, as illustrated in FIG. 4 The loop 48 remains exposed around the tensioning band 30 and can be used to pull the rod 34 from the pocket 44 if pushed beyond the pocket opening.

While one or more embodiments of the present invention have been described in detail, it should be apparent that modifications and variations thereto are possible, all of which fall within the true spirit and scope of the invention. Therefore, the foregoing is intended only to be illustrative of the principles of the invention. Further, since numerous modifications and changes will readily occur to those skilled in the art, it is not intended to limit the invention to the exact construction and operation shown and described. Accordingly, all suitable modifications and equivalents may be included and considered to fall within the scope of the invention, defined by the following claim or claims.

What is claimed is:

1. A tourniquet belt comprising:
a belt strap comprising an inner layer and an outer layer;
a tensioning band disposed between the inner and outer layers of the belt strap and fastened at each end to the belt strap;
a belt buckle at one end of the belt strap that engages with a free end of the belt strap to secure the belt strap around a user's waist;
a fastening mechanism for securing the belt strap in an inverted orientation around an appendage for use as a tourniquet;
an opening in the inner layer of the belt strap exposing a portion of the tension band when the belt strap is secured around the appendage;
a tensioning rod configured for insertion beneath the exposed portion of the tensioning band and operable to tighten the tensioning band when the tensioning rod is rotated by the user; and
a pocket disposed on the inner layer for storing the tensioning rod when the tourniquet belt is not being used as a tourniquet.

2. The tourniquet belt of claim 1, further comprising a lanyard attached at each end to the tensioning rod and passing beneath the exposed portion of the tensioning band to retain the tensioning rod.

3. The tourniquet belt of claim 1, further comprising a retention tab attached to the inner layer of the belt strap for engagement with the tightening rod after the tourniquet belt is tightened around the appendage to maintain the tension on the tensioning band.

4. The tourniquet belt of claim 3, further comprising one or more elastic keepers attached to the inner layer to hold the retention tab flat against the inner layer when the tourniquet belt is used as a belt.

5. The tourniquet belt of claim 1, wherein the fastening mechanism comprises a hook and loop faster attached to the inner layer of the belt strap.
